(19) United States
(12) Patent Application Publication
(10) Pub. No.: US 2023/0119668 A1
(43) Pub. Date: Apr. 20, 2023

(54) PREDICTIVE SENSOR SYSTEM, METHOD, AND COMPUTER PROGRAM PRODUCT

(71) Applicant: Murata Manufacturing Co., Ltd., Nagaokakyo-shi (JP)

(72) Inventor: Takahiro YAMAZAKI, Nagaokakyo-shi (JP)

(73) Assignee: Murata Manufacturing Co., Ltd., Nagaokakyo-shi (JP)

(21) Appl. No.: 18/083,591

(22) Filed: Dec. 19, 2022

Related U.S. Application Data
(63) Continuation of application No. PCT/JP2022/011921, filed on Mar. 16, 2022.

(30) Foreign Application Priority Data
Apr. 30, 2021 (JP) 2021-077442

(51) Int. Cl. G06N 5/022

(52) U.S. Cl. CPC G06N 5/022

(57) ABSTRACT

A predictive sensor system has a memory that collects time series data from a sensor that detects a parameter of an atmosphere in an inhabitable space. Circuitry implements a trained predictive sensor model and generates predicted sensor measurement data for a future segment of time. The trained predictive sensor model includes a first trained model and a second trained model. The first trained model is more suitable for forecasting the predicted data in a first forecasting segment than the second trained model. The second trained model is more suitable for forecasting the predicted data in a second forecasting segment than the first trained model wherein the second forecasting segment comes later than the first forecasting segment. The circuitry also forecasts the predicted data in another time segment between the first forecasting segment and the second forecasting segment based on the time series data and the trained predictive sensor model.

### CROSS-REFERENCE TO RELATED APPLICATIONS

The present application is a continuation application of International Patent Application No. PCT/JP2022/011921, filed Mar. 16, 2022, which claims priority to Japanese patent application No. 2021-077442, filed Apr. 30, 2021, the entire contents of each of which being incorporated herein by reference.

### TECHNICAL FIELD

The present disclosure relates to a predictive sensor system, a predictive sensing method, and a predictive sensor computer program product, each of which supplements sensor measurements with predicted sensor levels in a change of time series data. Moreover, the present disclosure relates to a predictive sensor system that provide predicted changes in time series sensor data, where the system senses a characteristic of a physical environment.

### BACKGROUND ART

In general, a technique for forecasting data that change as time passes (hereinafter, also referred to as “time series data”) is known in the art. Japanese Unexamined Patent Application Publication No. 2016-126718 (Patent Document 1) discloses a forecasting device that forecasts changes in time series data that are predicted to occur in the future by analyzing past time series data in every time segment. The forecasting device disclosed in the Patent Document 1 is configured in such a manner as to forecast changes of time series data after a certain time segment by selecting a single kernel function based on an analysis result of past time series data in that time segment and applying support vector regression to the selected kernel function.

### CITATION LIST

### Patent Document

Patent Document 1: Japanese Unexamined Patent Application Publication No. 2016-126718

### SUMMARY

### Technical Problems

As one example, the forecasting device disclosed in the Patent Document 1 is configured to select a single kernel function for a single time segment to be analyzed. Thus, with regard to a forecasting segment to which the selected kernel function is applicable, this forecasting device can forecast change of time series data with a high degree of accuracy. However, as recognized by the present inventor, the forecasting device cannot take into consideration the analysis result of a forecasting segment whose length exceeds the applicable range of the selected kernel function. Therefore, there is a risk of not being able to accurately forecast the change of time series data over a long period of time.

One non-limiting aspect of the present disclosure is to resolve such issues, and provide various techniques for accurately predict changes in time series data of sensor(s) over long periods of time.

### Solutions to Problems

As one non-limiting aspect of the present disclosure, a predictive sensor system according to an aspect of the present disclosure includes a non-transitory computer readable memory that stores time series data and circuitry that is configured by software to augment the sensor measurements to provide predictive sensor readings. The circuitry executes the computer readable instructions to implement a trained predictive sensor model that predicts “predicted time series data” (also referred to herein as “predicted data”) from earlier time series data collected from a sensor. The trained predictive sensor model includes a first trained model and a second trained model. The first trained model is more suitable for forecasting the predicted data in a first forecasting segment than the second trained model. The second trained model is more suitable for forecasting the predicted data in a second forecasting segment than the first trained model. The second forecasting segment comes later in time than the first forecasting segment. The circuitry runs the trained model to also generate predicted data in another segment of time in between the first forecasting segment and the second forecasting segment based on the time series data and the trained predictive sensor model.

A predictive sensor method according to another aspect of the present disclosure stores in memory time series data from a sensor(s) and applies the time series data to a trained predictive sensor model implemented in circuitry that is configured by computer readable instructions that are executed by the circuitry. The process further includes running the trained predictive sensor model on the time series data to generate predicted data, the predicted data being data that is predicted sensor measurement data during a future segment of time. The first trained model is trained to more closely match a portion of the predicted data in a first forecasting segment of time than the second trained model. The second trained model is trained to more closely match another portion of the predicted data in a second forecasting segment of time than the first trained model, the second forecasting segment of time being later in time than the first forecasting segment of time. Also, the method includes forecasting an additional portion of the predicted data with the trained predictive sensor model in another segment of time in between the first forecasting segment of time and the second forecasting segment of time based on the time series data and the trained predictive sensor model.

A non-transitory computer program product according to another aspect of the disclosure includes computer readable instructions stored therein that when executed by a processing circuitry cause the processing circuitry to implement a method as discussed above.

### Advantageous Effects

According to the predictive sensor system, the predictive sensing method, and the predictive sensing computer program product of the present disclosure, it becomes possible to forecast the predicted data in the segment in between the first forecasting segment and the second forecasting segment using the predictive sensor model including the first model suitable for forecasting the predicted data of the time series data in the first forecasting segment and the second model suitable for forecasting the predicted data of the time series data in the second forecasting segment that comes later than the first forecasting segment. Because of this, the predictive sensor system, the predictive sensing method, and the predictive sensing computer program product of the present disclosure not only enable forecasting of the change of the time series data provided by a sensor(s) in the first forecasting segment using the first model but also enable forecasting the change of the time series data in the segment in between the first forecasting segment and the second forecasting segment using the predictive sensor model, which includes the first model and the second model. This enables to accurately forecast the change of the time series data over a long period of time.

### BRIEF DESCRIPTION OF DRAWINGS

FIG. 1 is a diagram for describing an exemplary application of a predictive sensor system according to an embodiment 1.

FIG. 2 is a diagram illustrating a configuration of the predictive sensor system according to the embodiment 1.

FIG. 3 is a diagram illustrating change of time series data of sensor data forecasted by the predictive sensor system according to the embodiment 1.

FIG. 4 is a diagram for describing timings of processes of the predictive sensor system according to the embodiment 1.

FIG. 5 is a diagram for describing a parameter adjustment of a first model in the predictive sensor system according to the embodiment 1.

FIG. 6 is a diagram for describing a parameter adjustment of a second model in the predictive sensor system according to the embodiment 1.

FIG. 7 is a diagram illustrating a displaying form of a display of the predictive sensor system according to the embodiment 1.

FIG. 8 is a flowchart relating to processes to be executed by a control device of the predictive sensor system according to the embodiment 1.

FIG. 9 is a flowchart relating to other processes to be executed by the control component of the predictive sensor system according to the embodiment 1.

FIG. 10 is a diagram illustrating a configuration of a predictive sensor system according to a second embodiment.

FIG. 11 is a diagram for describing timings of processes of a predictive sensor system according to a third embodiment.

FIG. 12 is a diagram illustrating change of time series data of a sensor(s) forecasted by a predictive sensor system according to a fourth embodiment.

FIG. 13 is a diagram of computer-based circuitry and other devices that may cooperate to provide the processing circuitry used to execute the processor-based operations described herein.

### DESCRIPTION OF EMBODIMENTS

Hereinafter, embodiments of the present disclosure will be described in detail with reference to the drawings. Note that the same reference numerals are assigned to the same or corresponding portions in the drawings, and description thereof will not be repeated.

### Embodiment 1

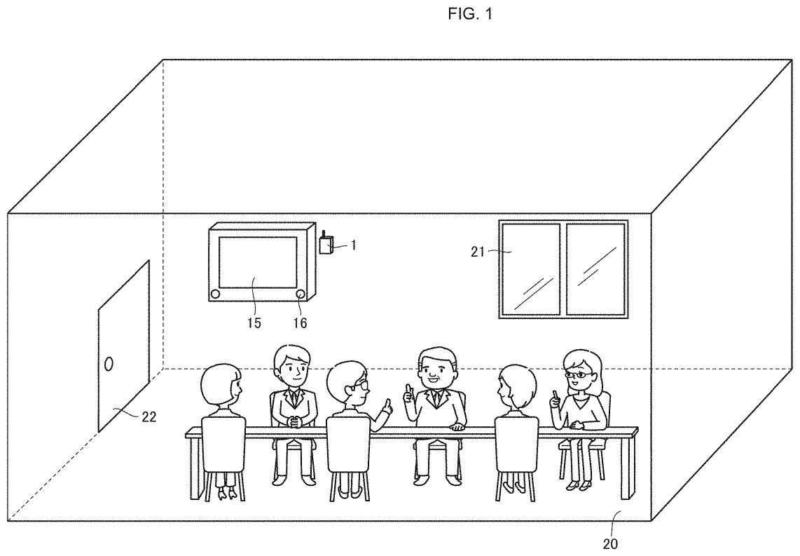

Referring to FIG. 1 to FIG. 9, a predictive sensor system 1 (sometimes called a forecasting device 1) according to the first embodiment is described.

(Exemplary Application)

FIG. 1 is a diagram for describing an exemplary application of the predictive sensor system 1 according to the first embodiment. The predictive sensor system 1 according to the embodiment 1 predicts changes of time series data by analyzing past time series data that have previously been obtained. The “time series data” mean a group of data items, from one or more sensors, in which a plurality of data items are arranged in a time series manner (i.e., sequential), and this plurality of data items is, for example, obtained in a time series manner (e.g., sequentially over time) from a single source such as, for example, a single sensor or the like.

For example, as illustrated in FIG. 1, in the case where people hold a meeting in an indoor space 20 where a window 21 and a door 22 are both closed, the concentration of carbon dioxide (hereinafter, also referred to as “CO2”) in the indoor space 20 can rise. In order to avoid a rise of CO2 concentration in the closed indoor space 20 beyond an unwelcomed level, it is necessary to expel some CO2 from the indoor space 20 by ventilating the open space in some way, such as opening the window 21 or the door 22, operating an exhaust fan, and/or changing a damper setting on a vent from time to time.

Therefore, by analyzing CO2 concentrations levels in the indoor space 20 over time, the predictive sensor system 1 according to the embodiment 1 is able to forecast (or predict—“predict” and “forecast” are used interchangeable herein)—a CO2 concentration at a time in the future after the CO2 concentration level has risen again (hereinafter, such predicted future levels are also referred to herein as “predicted data”). Moreover, the predictive sensor system 1 calculates a “forecasted time-to-reach” (which is a time at which an item of the predicted data is forecasted to reach a threshold value) and a “forecasted time-period-to-reach” (which is a period of time from a forecasting time point (forecasting time) to the forecasted time-to-reach, and triggers a computer based process to generate a notification to a user of the predictive sensor system 1 (in this example, a person who is holding the meeting) of at least one of the forecasted time-to-reach and the forecasted time-period-to-reach, which have been calculated. Alternatively, or in addition to the notification, the predictive sensor system 1 can generate and dispatch a signal to generate a sensory output to alert an occupant in the inhabitable space of a level of the parameter of the atmosphere that corresponds with the predicted sensor measurement data. The signal may be used to trigger an electronic device to perform an activity such as generate a warning signal and/or control an operation of an electronic device (e.g., motor to open/close a window, door, or vent damper, or even operate another device such as switch on a light and/or turn of a fan). The “forecasting time point” (forecasting time) is at least one of time points (time) in the period of time from the time point (time) at which the forecasting of the predicted data starts to the time point (time) at which a forecasted result is calculated.

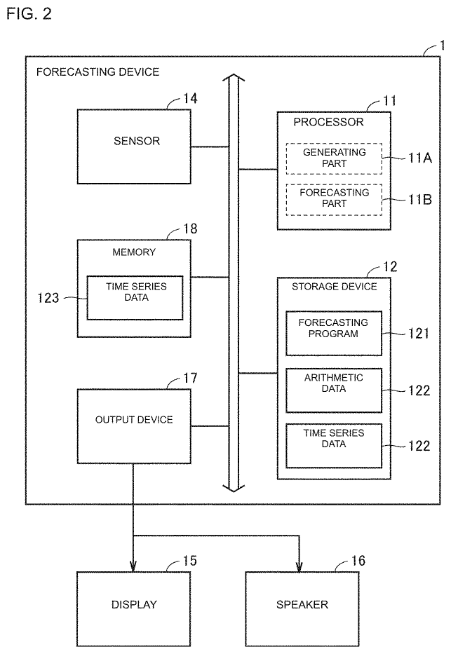

Specifically, in this non-limiting embodiment, the predictive sensor system 1 includes a sensor 14 as illustrated in FIG. 2, which will be described below. The sensor 14, in the example of FIG. 2, measures the CO2 concentration in the indoor space 20 periodically or according to a predetermined schedule, the timing for which is determined for example by a look up table or a scheduling process. By analyzing the change in the CO2 concentration obtained by the sensor 14, the predictive sensor system 1 forecasts the predicted data (which are predicted values of CO2 that occur in the future once the CO2 concentration changes) and calculates, based on a forecasted result, a forecasted time-to-reach at which a ventilation operation needs to be performed and/or a forecasted time-period-to-reach that is the period of time from the forecasting time point to the forecasted time-to-reach. The forecasting device 1 triggers a signal to an electronic device such as a notification process that causes a display driver to display an image on a display 15 for notifying a user of at least one of the forecasted time-to-reach and the forecasted time-period-to-reach, which have been calculated, and/or outputs a sound from a speaker 16 for notifying a user of at least one of the forecasted time-to-reach and the forecasted time-period-to-reach, which have been calculated. Alternatively, or in addition to the signaling, the forecasting device 1 may issue a command to actuate a motor that controllably opens or closes the window and/or door, or another device that can alter a characteristic of a physical environment such as a fan or vent, or a damper for the vent. Alternatively the command may control a switch that operates a light (to serve as another process for alerting occupants, such as by blinking the lights on/off) and/or fan.

This enables the predictive sensor system 1 to encourage a user to, and/or automatically, ventilate the indoor space 20 at proper timing using the forecasted result regarding the change of the CO2 concentration within the indoor space 20. Accordingly, a user can ventilate before the CO2 concentration in the closed indoor space 20 increases beyond a suitable level.

Note that the “predicted data” forecasted by the predictive sensor system 1 are not limited to the CO2 concentration but may alternatively be other data that change as time passes, such as humidity, light levels, temperature, or the like. The sensor 14 is not limited for measuring the CO2 concentration, but may alternatively measure other data that change as time passes, such as humidity, light level, temperature, or the like. Other gases may be monitored with sensors as well such as carbon monoxide, natural gas, propane, butane, methane and the like.

(Configuration of Forecasting Device)

FIG. 2 is a diagram illustrating a configuration of the predictive sensor system 1 (also referred to circuitry, which is software configurable) according to the embodiment 1. As illustrated in FIG. 2, the predictive sensor system 1 includes a processor 11, a memory 18, a storage device 12, a sensor 14, and an output device 17.

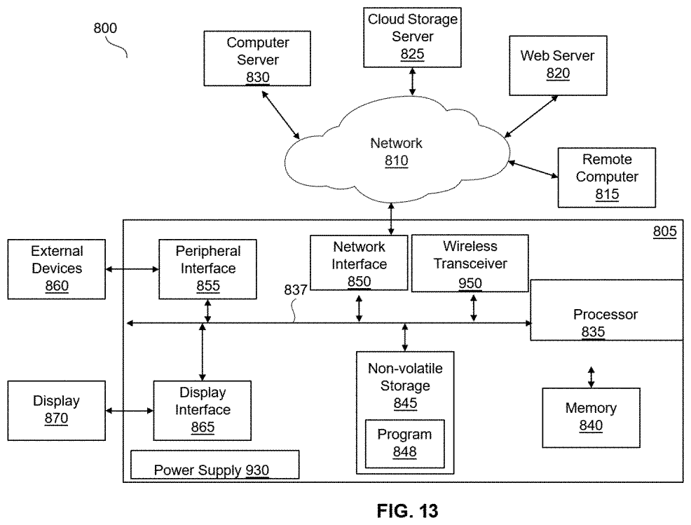

The processor 11 may be, for example, a processor based device such as a computer and is an arithmetic entity that executes various processes according to various computer readable programs. The processor 11 includes, for example, at least one of a CPU (Central Processing Unit), a FPGA (Field Programmable Gate Array), a GPU (Graphics Processing Unit), and a MPU (Multi Processing Unit). Note that the processor 11 may be made up of arithmetic circuitry (Processing Circuitry). A further example of the processor 11 and associated processing circuitry is more than one computer interconnected directly or wirelessly, such as via a local area network, or wireless network. One non-limiting example of the processor and associated processing circuitry is cloud computing resources that perform the calculations remotely and provide the results to the a local processor in the predictive sensor system 1. A greater discussion of the processing circuitry is provided below in reference to FIG. 13.

The memory 18 is an example of non-transitory computer readable medium and may be embodied as one or more volatile memory such as a DRAM (Dynamic Random Access Memory), a SRAM (Static Random Access Memory), or the like and/or a nonvolatile memory such as a ROM (Read Only Memory), a flash memory, or the like. The memory 18 temporarily stores time series data 123 obtained by the sensor 14. The time series data 123 are used at the time when the processor 11 forecasts an after-change CO2 concentration.

The storage device 12 is a computer readable storage medium that includes a nonvolatile memory such as an HDD (Hard Disk Drive), an SSD (Solid State Drive), or the like. The storage device 12 stores various programs (e.g., computer readable instructions) and data, such as a forecasting program 121 to be executed by the control device 11, arithmetic data 122 to be looked up by the processor 11, the time series data 123 obtained by the sensor 14, and the like. By using a computer(s), a server device, or distributed computer system, in which the processor 11, the storage device 12, and the output device 17 are implemented, it becomes possible for the change of time series data to be predicted based on user-request or user-setting.

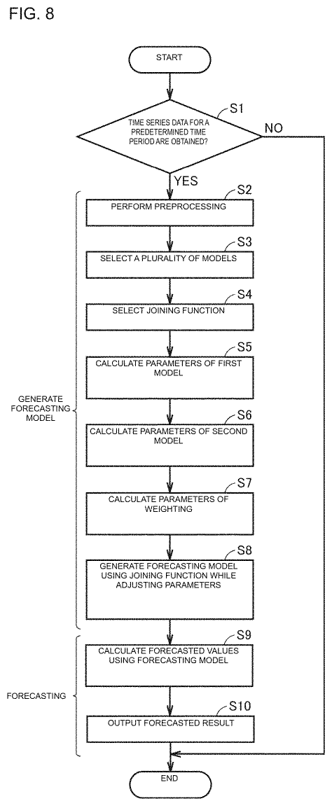

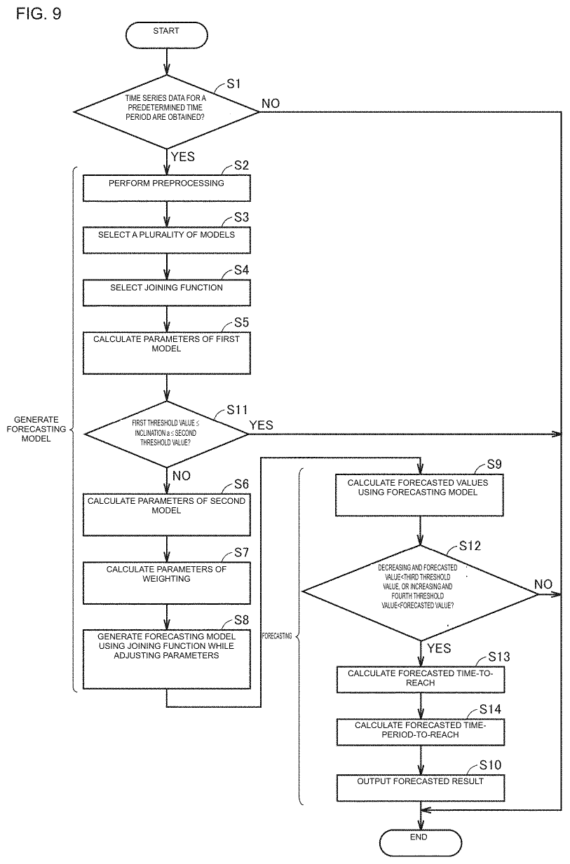

The forecasting program 121 includes programs (computer readable instructions) that specifies process steps (process flows illustrated in FIG. 8 and FIG. 9) of a generating part 11A and a forecasting part 11B, which are functional parts of the processor 11. Moreover, the terms “generating part” and “forecasting part” may be examples of generating circuitry and forecasting circuitry (or simply processing circuitry) and uses software instructions to configure the circuitry to implement the described processes and algorithms. The arithmetic data 122 are data to be used when processes according to the forecasting program 121 are executed and include data (for example, data relating to a first model, a second model, and a joining function, which will be described below, and the like) for generating a forecasting model that forecasts the predicted data.

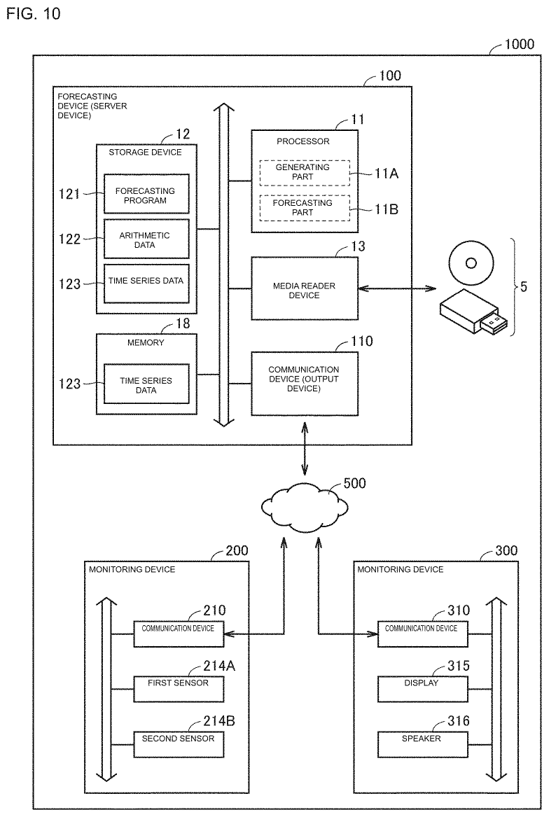

Note that the predictive sensor system 1 may store the forecasting program 121 and the arithmetic data 122 in the storage device 12 in advance or may obtain (e.g., download) the forecasting program 121 and the arithmetic data 122 via digital communication with a server device, accessible through the two-way interface circuitry shown as “output device” 17 in FIG. 2, although such server device is not expressly shown in FIG. 2. Further, it is not limited to the case where the processor 11 generates a forecasting model on the fly because the predictive sensor system 1 may store in the storage device 12 a forecasting model, or a variety of candidate forecasting models, prepared in advance. Moreover, as will be described below with using FIG. 10, the predictive sensor system 1 may further include a media reader device 13. Further, the predictive sensor system 1 may receive a removable disk 5, which is a non-transitory storage media, using a media reader device 13 and may obtain the forecasting program 121, the arithmetic data 122, and the forecasting model via the removable disk 5.

The sensor 14 measures time series data that change as time passes, such as CO2 (or other gas) concentration, humidity, light level, temperature, or the like, and outputs the obtained time series data to the storage device 12. The storage device 12 stores therein the time series data obtained from the sensor 14 as the time series data 123. Further, at the time when the processor 11 forecasts the predicted data, the time series data 123 are temporarily stored in the memory 18. Note that the predictive sensor system 1 may include not only the single sensor 14 but also a plurality of sensors. In that case, the plurality of sensors may respectively measure different kinds of time series data. For example, of the plurality of sensors, a first sensor may detect the CO2 concentration as the time series data, a second sensor may detect the humidity as the time series data, and a third sensor may detect temperature as the time series data. In that case, the predictive sensor system 1 may forecast the change of each group of time series data based on the time series data obtained by each of the first sensor, the second sensor, and the third sensor. Moreover, the plurality of sensors may each measure the same kind of time series data as another sensor. For example, of the plurality of sensors, each of a first sensor and a second sensor may detect CO2 concentration as the time series data, and a third sensor may detect temperature as the time series data. In that case, the predictive sensor system 1 may forecast the change of CO2 concentration based on the time series data of the CO2 concentration obtained by each of the first sensor and the second sensor and may forecast the change of temperature based on the temperature obtained by the third sensor.

The output device 17 (shown as an output device, but also serves as a two-way data interface that exchanges data, wirelessly or via a physical connection, between the predictive sensor system 1 and external circuitry) is connected to each of the display 15 and the speaker 16 using a wireless or wired connection. The output device 17 outputs data illustrating a forecasted result of the change of time series data calculated by the control device 11 to at least one of the display 15 and the speaker 16, or even generates a signal used to convey the forecasted result in a wired or wireless signal to another electronic device so the another electronic device can take action on the forecasted result (e.g., display, generate a warning, or perform a control operation such as control a motor, fan, or the like). One example of another electronic device is a smartphone, which itself can display the forecasting result to one or more occupants of the room.

Based on the data obtained from the output device 17, the display 15 displays various images (which may also be displayed on another device based on a signal sent from the output device 17) such as an image based on the forecasted result of the change of time series data calculated by the processor 11 and the like.

Based on the data obtained from the output device 17, the speaker 16 outputs various sounds such as a sound based on the forecasted result of the change of time series data calculated by the processor 11 and the like. Also, the output device 17 may send a signal that triggers a haptic device to cause a tactile sensation that is felt by a user.

Note that the display 15 and the speaker 16 are not necessarily separate constituent elements from the predictive sensor system 1. The predictive sensor system 1 may include at least one of the display 15 and the speaker 16, or another device such as the haptic device discussed above. Moreover, in the example of FIG. 1, the display 15 and the speaker 16 are installed in the indoor space 20. However, the display 15 and the speaker 16 (and/or haptic device) may be constituent elements included in a mobile terminal or personal computer (PC) owned by a user who is present in the room. For example, the predictive sensor system 1 may output the forecasted result to the mobile terminal or PC owned by the user using a near field communication, Bluetooth signaling, Wi-Fi signaling, and/or cellular transmissions such as SMS signal, or 5G communication, or the like, and the mobile terminal or PC may notify the user of the forecasted result obtained from the predictive sensor system 1 using the display 15 or the speaker 16 thereof (of the mobile terminal or PC).

The predictive sensor system 1 configured as described above stores the time series data 123 obtained by the sensor 14 in the storage device 12 or the memory 18 and forecasts the predicted data using the processor 11 based on the time series data 123 stored in the storage device 12 or the memory 18. Further, the predictive sensor system 1 notifies a user of information based on the forecasted result using at least one of the display 15 and the speaker 16.

The processor 11 executes the forecasting program 121 stored in the storage device 12 to implement a series of processes such as the ones described above by performing arithmetic operations using the arithmetic data 122 as need arises. More specifically, the processor 11 includes, as primary functional parts, the generating part 11A and the forecasting part 11B (which as discussed above may also be described as circuitry configured by computer readable instructions to implement generation circuitry or a generation process, and forecast circuitry or forecast process, respectively).

The generating part 11A generates a forecasting model (e.g., trained forecasting model, or trained forecasting model circuitry) for forecasting predicted data (e.g., after-change data). In this context the model may be “trained” by (1) selection of mathematical formula from among a candidate set of mathematical formulas, and/or (2) a recursive process, such as by training the model via applying data vectors that are applied to a convolutional neural network (CNN), such as in a machine learning operation. Moreover, the trained predictive sensor model trained by including a function (mathematical formula) representing a relationship between the elapsed time and the data that change as time passes, where the function is chosen by the processor to match predicted data with actual data within a predetermined tolerance (e.g., +/−10% over the time segment). A segment (a segment of time, or even a segment of successive sensor measurements, described as a forecasting segment) over which the predictive sensor system 1 predicts the change of time series data includes a first forecasting segment (a first segment of time) and a second forecasting segment (a second segment of time), which comes later than the first forecasting segment. The generating part 11A generates a single forecasting model by joining (adding) a first trained model suitable for forecasting the predicted data in the first forecasting segment and a second trained model suitable for forecasting the predicted data in the second forecasting segment. Here, the “suitable model” means, of a plurality of models, a model that has been selected/trained (such as trained by selection of a suitable mathematically defined model from a group of models, or mathematically defined equation(s), or defined through a recursive “learning” method such as a convolutional neural network that is trained on vectors of input data) that can reproduce actual time series data with the highest accuracy of the different candidate models. That is to say, the “suitable model” is a trained model that can minimize the difference between the CO2 concentrations actually obtained in a forecasting segment and the “after-change”, predicted, CO2 concentrations (predicted data) forecasted in the forecasting segment. The first model is more suitable for forecasting the predicted data in the first forecasting segment than the second model and can reproduce the actual time series data in the first forecasting segment more accurately than the second model. The second model is more suitable for forecasting the predicted data in the second forecasting segment than the first model and can reproduce the actual time series data in the second forecasting segment more accurately than the first model.

The forecasting part 11B forecasts (or in other words, circuitry that has been configured, by computer readable instructions, to forecast) the predicted data using a forecasting model generated by the generating part 11A (or generation circuitry). Information based on the forecasted result obtained by the forecasting part 11B is output to the display 15 or the speaker 16, or other device, by the output device 17.

Note that functionalities of the respective constituent elements included in the predictive sensor system 1, such as the processor 11, the memory 18, the storage device 12, the output device 17, and the like, may be housed in the sensor 14 itself. That is to say, the sensor 14 itself may serve as the predictive sensor system 1 (circuitry configured to forecast) in a form of edge computer and include the constituent elements such as the processor 11, the memory 18, the storage device 12, the output device 17, and the like.

(Specific Example of Forecasting of Change of Time Series Data by Forecasting Device)

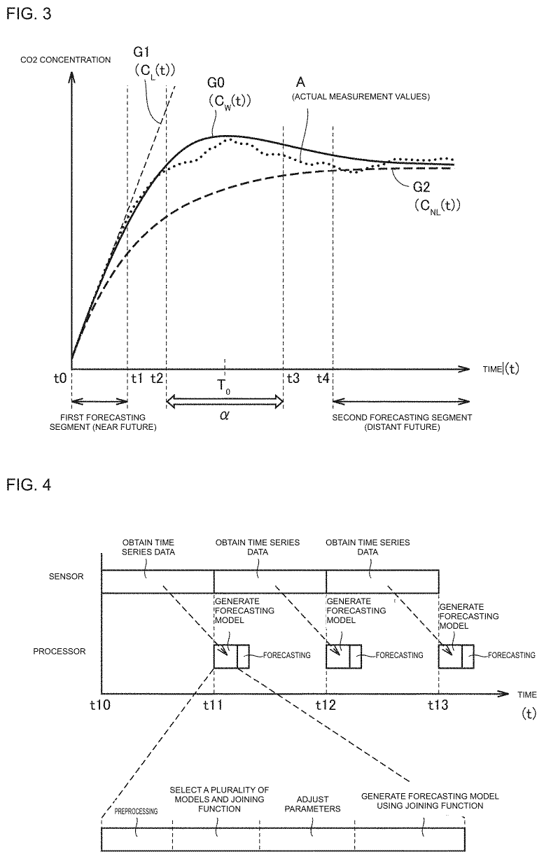

Referring to FIG. 3 to FIG. 6, the forecasting of the change of time series data by the predictive sensor system 1 is described. FIG. 3 is a diagram illustrating the change of time series data forecasted by the predictive sensor system 1 according to the embodiment 1.

FIG. 3 illustrates time series data of the CO2 concentration, where the vertical axis represents the CO2 concentration and the horizontal axis represents the time. The time point at which the predictive sensor system 1 forecasts change of time series data in future is defined as t0, and future timings after t0 are indicated as t1, t2, t3, and t4, as shown in FIG. 3.

To illustrate the forecasting segment of the predictive sensor system 1, the time segment between t0 and t1, in the example of FIG. 3, is represented as the first forecasting segment (near-future), and the time segment after t4 is represented as the second forecasting segment (distant, or more distant, future). That is to say, when viewed from the perspective of the forecasting time point t0 of the predictive sensor system 1, the first forecasting segment is a “near future” time segment compared with the second forecasting segment, which is a “distant future” time segment, as compared with the first forecasting time segment.

A dotted line plot “A” in FIG. 3 represents actual measurement values of the CO2 concentration that are actually obtained by the sensor 14 from t0 to t4 and thereafter. Referring to the plot A, in the first forecasting segment in near future from t0 to t1, the change of time series data with respect to the time change is represented as generally a linear form (y=mx+b, where y is the CO2 concentration level, m is the slope of the plot, x is the time axis value, and b is the y-intercept value), whereas in the second forecasting segment in distant future after t4, the change of time series data with respect to the time change is represented as a nonlinear form (not accurately representable as a y=mx+b plot).

For forecasting the change of time series data in the first forecasting segment, the first model, the linear model, is suitable. The first model, in this cases, is defined by a function (mathematical formula) representing a relationship between the elapsed time and the data that change as time passes. As the function is stable and characterizable mathematically, the model is a trained mathematical model. The function of the first model is suitable for forecasting time series data whose change with respect to the time change is represented as a linear form.

For example, a plot G1 represents a forecasted result of the change of time series data using the first model. In the first forecasting segment in near future, the plot G1 is represented as a linear form in such a manner as to be approximate to the plot A of the actual measurement values and generally matches the plot A. On the other hand, in the second forecasting segment in distant future, the plot G1 tends to separate from the plot A of the actual measurement values.

For forecasting the change of time series data in the second forecasting segment, the second model is suitable. The second model is defined by a function (mathematical formula) representing a relationship between the elapsed time and the data that change as time passes. The function of the second model is suitable for forecasting time series data whose change with respect to the time change is represented as a nonlinear form. In this context the second model is a trained mathematical model where the mathematical formula is identified as accurately matching observed characteristic measurements. Once again the generation of the mathematical expression may be implemented via regressive CNN operations via back-propagation of losses based on known losses from training vectors with ground truth values.

For example, a plot G2 represents a forecasted result of the change of time series data using the second model. In the second forecasting segment in distant future, the plot G2, shown in FIG. 3, is fairly represented as a nonlinear form in such a manner as to be approximate to the plot A of the actual measurement values and generally matches the plot A. On the other hand, in the first forecasting segment in near future, the plot G2 tends to separate from the plot A of the actual measurement values.

At t0, the predictive sensor system 1 generates a forecasting model to be used for forecasting the change of time series data in future after t0 by joining (e.g., concatenating) the first model and the second model that have the characteristics described above. A plot G0 represents a forecasted result of the change of time series data using the forecasting model. The plot G0 generally matches the plot A of the actual measurement values in both the first forecasting segment and the second forecasting segment.

FIG. 4 is a diagram for describing timings of processes of the predictive sensor system 1 according to the first embodiment, embodiment 1. In FIG. 4, timings of processes performed by the sensor 14 and timings of processes performed by the processor 11 are illustrated.

As illustrated in FIG. 4, when the sensor 14 obtains time series data over a time period from t10 to t11, the processor 11 generates the forecasting model after t11 based on the time series data (time series data from t10 to t11) and forecasts the change of time series data after t11 using the generated forecasting model.

When the sensor 14 obtains time series data over a time period from t11 to t12, the control device 11 generates another forecasting model after t12 based on the time series data (time series data from t11 to t12) and forecasts the change of time series data after t12 using the newly generated forecasting model.

When the sensor 14 obtains time series data over a time period from t12 to t13, the control device 11 re-generates the forecasting model again after t13 based on the time series data (time series data from t12 to t13) and forecasts the change of time series data after t13 using the re-generated forecasting model.

As described above, the predictive sensor system 1 periodically (or according to scheduled or predetermined times, or according to a certain number of observed measurements) generates the forecasting model for each of predetermined time segments based on the time series data obtained by the sensor 14 and forecasts, using the forecasting model, the change of time series data in a forecasting segment that follows the time segment over which the time series data are obtained. The predictive sensor system 1 repeats such processes in each time segment.

The processes for generating the forecasting model includes a step of performing preprocessing, a step of selecting a plurality of models, a step of selecting a joining function, a step of adjusting parameters, and a step of generating the forecasting model using the joining function.

Specifically, as illustrated in FIG. 4, when time series data are obtained from the sensor 14, the processor 11 first performs preprocessing on the time series data. For example, as the preprocessing, the processor 11 performs interpolation of missing data in the time series data, removal of data items (outliers) that greatly deviate from other data items in a plurality of data items included in the time series data, removal of noise appeared in the time series data, transformation using a function (operator), and the like. By performing preprocessing that fits characteristics of the obtained time series data, the processor 11 transforms the obtained time series data into data suitable for generating the forecasting model.

Note that for the removal of the noise included in the preprocessing, a moving average method, smoothing that uses a state space model, a low pass filter, and the like are available, and may be used individually or in combination. For the transformation using a function (operator) included in the preprocessing, Fourier conversion, compression transformation of feature values that uses principal component analysis, and the like are available and may be used individually or in combination.

The processor 11 is able to perform highly accurate forecasting without being influenced by noise by performing preprocessing such as the anomalous ones described above on the time series data obtained by the sensor 14. On the other hand, in the case where a highly accurate forecasting is possible using the time series data obtained by the sensor 14 without any change, the processor 11 may omit the preprocessing step such as the one described above and may generate the forecasting model directly using the time series data obtained by the sensor 14.

After performing the preprocessing, the processor 11 selects a plurality of candidate models to be used for generating the forecasting model. For example, in the example illustrated in FIG. 3, the processor 11 selects, from a larger set of models, the first model suitable for forecasting in the first forecasting segment, which is represented by the graph G1, and the second model suitable for forecasting in the second forecasting segment, which is represented by the graph G2. Note that data relating to a plurality of models including the first model and the second model are included in the arithmetic data 122 stored in the storage device 12.

The first model includes a linear function (mathematical formula) represented by the following equation (1). That is to say, the first model is a linear model in which the change of time series data with respect to the time change is represented as a linear form (previously described as y=mx+b).

[Math 1]

C_{L}(t)=at+b (1)

In the equation (1), a is a parameter representing the inclination (or slope), and b is a parameter representing the y-axis intercept.

The second model includes a nonlinear function (mathematical formula) represented by the following equation (2). That is to say, the second model is a nonlinear model in which the change of time series data with respect to the time change is represented as a nonlinear form.

$$\begin{matrix}[ Math 2 ] & \\ {C}_{NL} ( t ) - {C}_{0} = \frac{G}{Q} ( 1 - {e}^{- \frac{Q}{V} t} ) & ( 2 )\end{matrix}$$

In the equation (2), C₀ is a parameter representing the CO2 concentration in the indoor space 20 at a certain time t=0. G is a parameter representing a produced amount of CO2 produced in the indoor space 20. Q is a parameter representing a ventilated amount of CO2 expelled from the indoor space 20. V is a parameter representing the volume of the indoor space 20.

Note that the processor 11 may respectively select predetermined models for the first model and the second model from the candidate models that have been stored in advance. For example, by analyzing time series data obtained in advance, a designer (or a computer-based processor that records candidate models based on past observed time series data segments) of the predictive sensor system 1 may store a predetermined model X (for example, the model represented by the equation (1)) in the storage device 12 as the first model suitable for the first forecasting segment. Moreover, by analyzing time series data obtained in advance, a designer (or the computer-based processor) of the predictive sensor system 1 may store a predetermined model Y (for example, the model represented by the equation (2)) in the storage device 12 as the second model suitable for the second forecasting segment. Further, at the time of generating the forecasting model, the processor 11 may select the model X stored in the storage device 12 as the first model (e.g., using a least squares fit analysis) and may select the model Y stored in the storage device 12 as the second model.

Alternatively, of plural kinds of models, the processor 11 may select models that correspond to the respective ones of the first model and the second model based on a result of analyzing the obtained time series data without predetermining models that correspond to the respective ones of the first model and the second model. For example, a designer of the predictive sensor system 1 (or the computer-based processor) may store the plural kinds of models in the storage device 12. Further, at the time of generating the forecasting model, the processor 11 may analyze the obtained time series data and based on the analysis result, may select at least one of the plural kinds of models stored in the storage device 12 as the first model and at least one of the plural kinds of models stored in the storage device 12 as the second model. Note that the processor 11 may select, as the second model, a model different from the model that has been selected as the first model.

As illustrated in FIG. 4, after a plurality of models is selected, the processor 11 selects the joining function for joining the selected plurality of models. Note that data relating to the joining function are included in the arithmetic data 122 stored in the storage device 12.

In the example illustrated in FIG. 3, the processor 11 selects a hyperbolic function represented by the following equation (3) as the joining function.

$$\begin{matrix}[ Math 3 ] & \\ {C}_{W} ( t ) = {C}_{L} ( t ) * ( \frac{1 - tanh ( \frac{t - {T}_{0}}{α} )}{2} ) + {C}_{NL} ( t ) * ( \frac{1 + tanh ( \frac{t - {T}_{0}}{α} )}{2} ) & ( 3 )\end{matrix}$$

In the equation (3), C_{L}(t) is the function of the first model represented by the equation (1). C_{NL}(t) is the function of the second model represented by the equation (2).

At the time of joining the first model and the second model, the processor 11 weights the first model and the second model. In the equation (3), α and T₀ are parameters relating to weighting at the time of joining the first model and the second model. The “parameters relating to weighting” determine, of a value obtained by the first model and a value obtained by the second mode, which value should preferentially be used. In the equation (3), until the time t reaches the time T0, CL(t) is multiplied by a larger value compared with the CNL(t) in such a way that the value of CL(t) is preferentially used over the value of CNL(t).

Specifically, as illustrated in FIG. 3, a is a parameter representing the time period required to switch the first model to the second model. As a becomes smaller, the time period required to switch the model from the first model to the second model becomes shorter. As a becomes larger, the time period required to switch the model from the first model to the second model becomes longer.

T₀ is a parameter representing the time of an intermediate point in the period of time during which the model is switched from the first model to the second model. As T₀ becomes smaller, the time period from the forecasting time point t0 to the switching of the model becomes shorter. As T₀ becomes smaller, the time period from the forecasting time point t0 to the switching of the model becomes shorter.

Note that the processor 11 may predetermine a joining function and may select the predetermined joining function. For example, by analyzing time series data obtained in advance, a designer of the predictive sensor system 1 may store a predetermined joining function (for example, the hyperbolic function represented by the equation (3)) in the storage device 12. Further, at the time of generating the forecasting model, the processor 11 may select the joining function stored in the storage device 12.

Alternatively, of plural kinds of joining functions, the processor 11 may select a joining function to be employed based on a result of analyzing the obtained time series data without predetermining the joining function. For example, a designer of the predictive sensor system 1 (or the computer-based processor) may store plural kinds of joining functions such as a joining function A to a joining function E in the storage device 12. Further, at the time of generating the forecasting model, the processor 11 may analyze the obtained time series data and based on the analysis result, may select at least one of the plural kinds of joining functions stored in the storage device 12.

As illustrated in FIG. 4, after selecting the joining function, the processor 11 adjusts the parameters. For example, in the example illustrated in FIG. 3, the processor 11 adjusts the parameters (a, b) of the first model represented by the equation (1). The processor 11 adjusts the parameters (C₀, G/Q, Q/V) of the second model represented by the equation (2). The processor 11 adjusts the parameters (a, T₀) of the joining function represented by the equation (3).

Note that the processor 11 may adjust all of the parameters in each of the joining function and a plurality of models or may adjust the parameters of at least one of the joining function and the plurality of models. For example, in the embodiment 1, a designer of the predictive sensor system 1 (or the computer-based processor) predetermines values of the parameters (α, T₀) of the joining function by analyzing time series data obtained in advance. Specifically, a designer of the predictive sensor system 1 (or the computer-based processor) stores 1000 seconds as the value of a and 600 seconds as the value of T₀ in the storage device 12 in advance. Further, the processor 11 is configured in such a way that the values of a and T₀ stored in the storage device 12 are used as the parameters of the joining function.

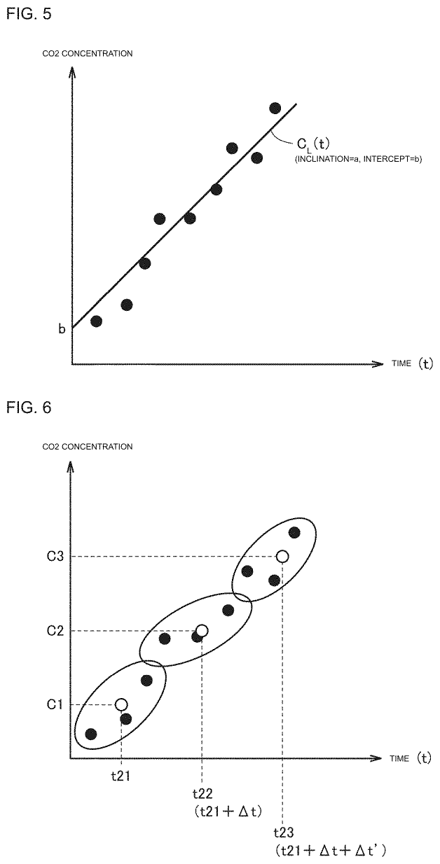

FIG. 5 is a diagram for describing a parameter adjustment of the first model in the predictive sensor system 1 according to the embodiment 1.

FIG. 5 illustrates time series data of the CO2 concentration, where the vertical axis represents the CO2 concentration and the horizontal axis represents the time. The processor 11 determines the parameters (a, b) of the first model represented by the equation (1) using a known least-square method.

Specifically, as illustrated in FIG. 5, the processor 11 extracts a predetermined number of most recent data items from the past time series data obtained from the sensor 14. Based on a plurality of extracted data items, the processor 11 calculates the parameters (a, b) of the first model using the least-square method.

Note that in the case where the forecasting time point is t11 in the example illustrated in FIG. 4, the predetermined number of data items extracted by the processor 11 includes, of the time series data obtained over the time period from t10 to t11, a data item obtained at the timing closest possible to the forecasting time point t11.

FIG. 6 is a diagram for describing a parameter adjustment of the second model in the predictive sensor system 1 according to the embodiment 1.

FIG. 6 illustrates time series data of the CO2 concentration, where the vertical axis represents the CO2 concentration and the horizontal axis represents the time. The processor 11 determines the parameters (C₀, G/Q, Q/V) of the second model represented by the equation (2) using computation of a known system of equations.

Specifically, as illustrated in FIG. 6, the processor 11 extracts a predetermined number of data items from the past time series data obtained from the sensor 14. The processor 11 divides a plurality of extracted data items in time series order for respective ones of a plurality of segments. The processor 11 calculates an average CO2 concentration and an average time for a plurality of data items belonging each segment.

For example, based on a plurality of data items belonging to the first segment, the processor 11 calculates C1 as the average CO2 concentration and t21 that corresponds to C1 as the average time. Based on a plurality of data items belonging to the second segment, the processor 11 calculates C2 as the average CO2 concentration and t22 that corresponds to C2 as the average time. Based on a plurality of data items belonging to the third segment, the processor 11 calculates C3 as the average CO2 concentration and t23 that corresponds to C3 as the average time. Moreover, the processor 11 calculates a difference Δt between the average time t21 of the plurality of data items belonging to the first segment and the average time t22 of the plurality of data items belonging to the second segment. The processor 11 calculates a difference Δt′ between the average time t22 of the plurality of data items belonging to the second segment and the average time t23 of the plurality of data items belonging to the third segment.

Here, the equation (2) can be represented by the following equation (4).

$$\begin{matrix}[ Math 4 ] & \\ {C}_{NL} ( t + 1 ) - {C}_{0} - \frac{G}{Q} = {e}^{- \frac{Q}{V}} ( {C}_{NL} ( t ) - {C}_{0} - \frac{G}{Q} ) & ( 4 )\end{matrix}$$

The processor 11 sets up the following system of equations (5) by using the equation (4) and the foregoing C1, C2, C3, Δt, and Δt′.

$$\begin{matrix}[ Math 5 ] & \\ {C}_{1} = {C}_{0} & ( 5 )\end{matrix}$$

$${C}_{2} - {C}_{0} - \frac{G}{Q} = {e}^{- \frac{Q}{V} Δ t} ( {C}_{1} - {C}_{0} - \frac{G}{Q} )$$

$${C}_{3} - {C}_{0} - \frac{G}{Q} = {e}^{- \frac{Q}{V} Δ {t}^{′}} ( {C}_{2} - {C}_{0} - \frac{G}{Q} )$$

Note that the processor 11 may alternatively set up the following system of equations (6) by using the equation (4) and the foregoing C1, C2, C3, Δt, and Δt′.

$$\begin{matrix}[ Math 6 ] & \\ {C}_{1} - {C}_{0} - \frac{G}{Q} = {e}^{\frac{Q}{V} Δ t} ( {C}_{2} - {C}_{0} - \frac{G}{Q} ) & ( 6 )\end{matrix}$$

$${C}_{2} - {C}_{0} - \frac{G}{Q} = {e}^{\frac{Q}{V} Δ {t}^{′}} ( {C}_{3} - {C}_{0} - \frac{G}{Q} )$$

$${C}_{3} = {C}_{0}$$

The processor 11 calculates the parameters (C₀, G/Q, Q/V) of the second model by solving the system of equations (5) or the system of equations (6).

As illustrated in FIG. 4, after the adjustment of the parameters, the processor 11 generates the forecasting model by joining a plurality of models using the joining function. For example, in the example illustrated in FIG. 3, the processor 11 generates the forecasting model represented by the graph G0 by joining the first model suitable for forecasting in the first forecasting segment represented by the graph G1 and the second model suitable for forecasting in the second forecasting segment represented by the graph G2 using the hyperbolic function represented by the equation (3). At that time, the processor 11 applies the values determined by the parameter adjustments to the parameters (a, b) of the first model, the parameters (C₀, G/Q, Q/V) of the second model, and the parameters (α, T₀) relating to the weighting.

Note that with regard to C₀ in the parameters of the second model, after adjusting each parameter, by replacing with the value of the CO2 concentration immediately before generating the forecasting model, it becomes possible to forecast the value reflecting the most recent state.

By using the forecasting model generated through steps such as the ones described above, the predictive sensor system 1 can predict the change of time series data in such a manner as to generally match the actual measurement values of the CO2 concentration actually obtained by the sensor 14.

Note that the processor 11 may adjust the parameters of each of the first model and the second model or may adjust the parameters of at least one of the first model and the second model. For example, the processor 11 may adjust the parameters (a, b) of the first model, while the parameters (C₀, G/Q, Q/V) of second model may be predetermined. Alternatively, the processor 11 may adjust the parameters (C₀, G/Q, Q/V) of the second model, while the parameters (a, b) of the first model may be predetermined.

(Displaying Form of Display)

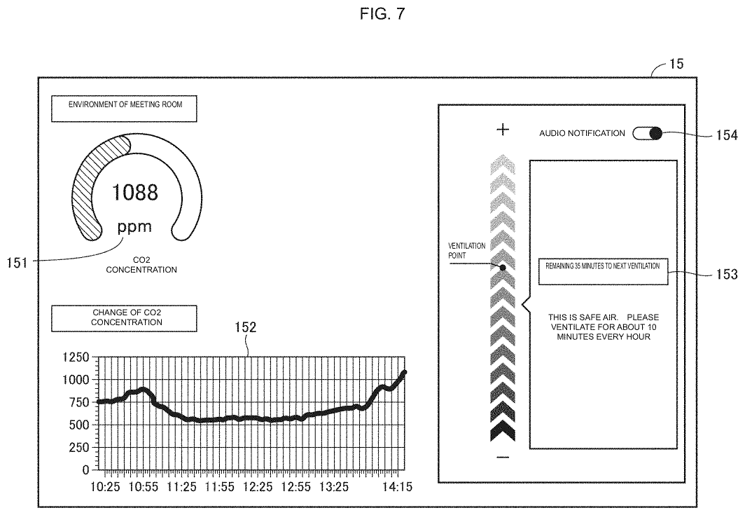

FIG. 7 is a diagram illustrating a displaying form of the display 15 of the predictive sensor system 1 according to the embodiment 1. The processor 11 causes the display 15 to display an image based on the forecasted result by outputting the forecasted result to the display 15 using the output device 17.

For example, in the case of the example of FIG. 1, as illustrated in FIG. 7, the display 15 displays an image relating to the CO2 concentration in the indoor space 20. The image displayed on the display 15 includes an icon 151 illustrating the current CO2 concentration in the indoor space 20, a graph 152 illustrating the change of the CO2 concentration in the indoor space 20, an icon 153 illustrating a remaining time period (the forecasted time-period-to-reach) until ventilation of the indoor space 20 becomes necessary, and an icon 154 for enabling audio notification of the forecasted result using the speaker 16.

Note that a user may enable or disable the audio notification by applying a touch operation on the icon 154 or may enable or disable the audio notification by operating the icon 154 using a tool such as a mouse or the like, which is not illustrated in the drawing.

As described using FIG. 4, the predictive sensor system 1 calculates the remaining time period until ventilation of the indoor space 20 becomes necessary by periodically generating the forecasting model for each of predetermined time segments and forecasting the change of the CO2 concentration in the indoor space 20 using the generated forecasting model. Further, the predictive sensor system 1 notifies a user of the calculated remaining time period using the icon 153.

As described above, the predictive sensor system 1 outputs, to the display 15, the forecasted time-period-to-reach from the forecasting time point, at which the predicted data are forecasted, to the forecasted time-to-reach, at which an item of the predicted data is forecasted to reach a threshold value. Note that the predictive sensor system 1 may output to the display 15 not only the forecasted time-period-to-reach but also the forecasted time-to-reach.

Moreover, the predictive sensor system 1 may forecast the predicted data for each future time at a first forecasting time point and calculate a first forecasted time-to-reach at which an item of the predicted data is forecasted to reach a threshold value, and then again forecast the predicted data for each future time at a second forecasting time point, which comes later than the first forecasting time point, and calculate a second forecasted time-to-reach at which an item of the predicted data is forecasted to reach the threshold value. Further, in the case where the time period between the first forecasted time-to-reach and the second forecasted time-to-reach is greater than or equal to a predetermined time period, the predictive sensor system 1 may output a signal to specify that the forecasted time-to-reach calculated at the first forecasting time point has changed. For example, the predictive sensor system 1 may notify a user of the change of the forecasted time-to-reach using the display 15 by outputting to the display 15 a signal to specify that the forecasted time-to-reach has changed. Note that the “threshold value” described above is an index value indicating that the ventilation is necessary and, for example, corresponds to a fourth threshold value, which will be described below.

This enables the predictive sensor system 1 to encourage a user to ventilate at proper timing before the CO2 concentration in the indoor space 20 increases.

(Processes by Forecasting Device)

FIG. 8 is a flowchart relating to processes to be executed by the processor 11 of the predictive sensor system 1 according to the embodiment 1. The processor 11 periodically executes the processes of the flowchart illustrated in FIG. 8 by executing the forecasting program 121 stored in the storage device 12. Note that S2 to S8 in this drawing correspond to the processes of the generating part 11A of the processor 11. S9 to S10 in this drawing correspond to the processes of the forecasting part 11B of the processor 11. In this drawing, “S” is used as an abbreviation of “STEP”.

As illustrated in FIG. 8, the processor 11 determines whether time series data of a predetermined time period are obtained or not (S1). For example, as illustrated in FIG. 4, in the case where the forecasting time point is t11, the processor 11 determines whether time series data are obtained over the time period from t10 to t11 or not. When the time series data of the predetermined time period are not obtained (NO in S1), the processor 11 ends the process of the present flowchart.

On the other hand, when the time series data of the predetermined time period are obtained (YES in S1), the processor 11 proceeds to processes for generating the forecasting model. Specifically, first, the processor 11 performs preprocessing (S2).

The processor 11 selects a plurality of models (S3). For example, the processor 11 selects a linear model represented by the equation (1) as the first model and selects a nonlinear model represented by the equation (2) as the second model.

The processor 11 selects a joining function for joining the selected plurality of models (S4). For example, the processor 11 selects a hyperbolic function represented by the equation (3) as the joining function.

The processor 11 calculates the parameters of the first model (S5). For example, the processor 11 calculates the parameters (a, b) of the first model represented by the equation (1). Note that as described in FIG. 5, the parameters (a, b) of the first model can be calculated by using the least-square method.

The processor 11 calculates the parameters of the second model (S6). For example, the processor 11 calculates the parameters (C₀, G/Q, Q/V) of the second model represented by the equation (2). Note that as described in FIG. 6, the parameters (C₀, G/Q, Q/V) of the second model can be calculated by using the system of equations.

The processor 11 calculates the parameters relating to the weighting (S7). For example, the processor 11 calculates the parameters (α, T₀) of the hyperbolic function represented by the equation (3). Note that as described above, the parameters (α, T₀) relating to the weighting are predetermined by a designer of the predictive sensor system 1, and thus the processor 11 uses the values of a and T₀ stored in the storage device 12 as the parameters relating to the weighting.

The processor 11 generates the forecasting model by joining the plurality of models using the joining function (S8). For example, the processor 11 generates the forecasting model by joining the first model represented by the equation (1) and the second model represented by the equation (2) using the hyperbolic function represented by the equation (3). At that time, the processor 11 applies the values determined by the parameter adjustments to the parameters (a, b) of the first model, the parameters (C₀, G/Q, Q/V) of the second model, and the parameters (α, T₀) relating to the weighting.

After generating the forecasting model, the processor 11 proceeds to processes for forecasting the change of time series data using the generated forecasting model. Specifically, the processor 11 calculates forecasted values using the forecasting model (S9). For example, as illustrated in FIG. 3, in the case where the present time point is t0, the processor 11 calculates values of the predicted data (forecasted values) in the forecasting segment including the first forecasting segment and the second forecasting segment. Note that the processor 11 may calculate forecasted values of all the time series data to be obtained in the forecasting segment or may calculate only some of the forecasted values of all the time series data to be obtained in the forecasting segment.

The processor 11 outputs the forecasted result including the calculated forecasted values to at least one of the display 15 and the speaker 16 (S10). After that, the processor 11 ends the present processes.

Next, referring to FIG. 9, other processes to be executed by the predictive sensor system 1 are described. FIG. 9 is a flowchart relating to other processes to be executed by the processor 11 of the predictive sensor system 1 according to the embodiment 1. In the following description, of the processes illustrated in FIG. 9, only the parts different from the processes illustrated in FIG. 8 are described. The predictive sensor system 1 may be configured to execute the processes illustrated in FIG. 8 or may be configured to execute the processes illustrated in FIG. 9.

The processor 11 periodically executes the processes of the flowchart illustrated in FIG. 9 by executing the forecasting program 121 stored in the storage device 12. In this drawing, “S” is used as an abbreviation of “STEP”.

As illustrated in FIG. 9, the processor 11 executes the processes of S1 to S4 and further executes the process of S11 after calculating the parameters of the first model in S5. In the process of S11, the processor 11 approximately determines how the time series data change. In the case where the inclination a is greater than or equal to a first threshold value and is less than or equal to a second threshold value, where the first threshold value has a value less than or equal to 0 and the second threshold value has a value greater than or equal to 0, the processor 11 determines that the time series data are in a stationary state and ends the present process flow without generating the forecasting function or performing the forecasting. This enables to omit unnecessary calculations.

Specifically, of the calculated parameters, the processor 11 determines whether or not the inclination a is greater than or equal to the first threshold value and is less than or equal to the second threshold value (S11). For example, in the case where the first threshold value is a value less than or equal to 0 and the inclination a is less than the first threshold value, that is, in the case where the inclination a has a minus value, the graph of the linear model represented by the equation (1) is negatively sloped as time passes. In this case, the time series data decrease as time passes. For example, in the case where the second threshold value is a value greater than or equal to 0 and the inclination a exceeds the second threshold value, that is, in the case where the inclination a has a plus value, the graph of the linear model represented by the equation (1) is positively sloped as time passes. In this case, the time series data increase as time passes.

In the case where the inclination a is greater than or equal to the first threshold value and is less than or equal to the second threshold value (YES in S11), that is, in the case where the graph of the linear model represented by the equation (1) is in the stationary state, the processor 11 ends the present processes.

On the other hand, in the case where the inclination a is less than the first threshold value or exceeds the second threshold value (NO in S11), the processor 11 generates the forecasting model by executing the processes of S6 to S8.

Next, after calculating the forecasted values in S9 using the forecasting model, the processor 11 executes the process of S12. In the processes of S12 and after, using the calculated forecasted values, the processor 11 can calculate a required time period (forecasted time-period-to-reach) until an item of time series data reaches a reference value. An example in which the CO₂ concentration is used as the time series data is described. For example, in the case where the CO2 concentration gradually is decreasing due to ventilation, the processor 11 calculates the forecast time-period-to-reach until the forecasted value reaches a third threshold value that is an index value indicating that adequate ventilation is provided. In the case where the CO2 concentration is increasing, the processor 11 calculates the forecast time-period-to-reach until the forecasted value reaches a fourth threshold value that is an index value indicating that ventilation is necessary. By causing the processor 11 to calculate and output the forecast time-period-to-reach such as the ones described above, it become possible to let a user know how much time is required to complete ventilation or how much time is required until ventilation becomes necessary. As the third threshold value and the fourth threshold value, values or index values set by laws and regulations of each country, values specified by a user, or the like can be used.

Specifically, the processor 11 determines whether or not the inclination a is less than the first threshold value and the forecasted value is less than the third threshold value or the inclination a exceeds the second threshold value and the forecasted value exceeds the fourth threshold value (S12). That is to say, the processor 11 determines whether or not the time series data are decreasing with the passage of time and the forecasted value is less than the third threshold value or the time series data are increasing with the passage of time and the forecasted value exceeds the fourth threshold value.

When it is not the case where the time series data are decreasing with the passage of time and the forecasted value is less than the third threshold value (NO in S12), for example, in the case where the CO2 concentration is decreasing during ventilation but the forecasted value is not expected to reach the third threshold value even if ventilation were to continue at the same pace, the processor 11 ends the present processes. Alternatively, when it is not the case where the time series data are increasing with the passage of time and the forecasted value is greater than or equal to the fourth threshold value (NO in S12), for example, in the case where the CO2 concentration is increasing but the forecasted value is not expected to reach the reference value at which ventilation is necessary (fourth threshold value), the processor 11 ends the present processes.

On the other hand, in the case where the time series data are decreasing with the passage of time and some of the forecasted values are less than the third threshold value (YES in S12), for example, in the case where the CO2 concentration is decreasing during ventilation and the forecasted value is forecasted to go below the third threshold value indicating that adequate ventilation is provided, the processor 11 proceeds to the process of S13. In the process of S13, the processor 11 calculates the forecasted time-to-reach at which the forecasted value of the time series data that are decreasing with the passage of time becomes less than the third threshold value.

Alternatively, in the case where the time series data are increasing with the passage of time and some of the forecasted values exceed the fourth threshold value (YES in S12), for example, in the case where the CO2 concentration is increasing and is forecasted to exceed the fourth threshold value at which ventilation is considered as necessary, the processor 11 proceeds to the process of S13. In the process of S13, the processor 11 calculates the forecasted time-to-reach at which the forecasted value of the time series data that are increasing with the passage of time becomes greater than the fourth threshold value.

The processor 11 calculates the forecasted time-period-to-reach that is the time period from the forecasting time point to the forecasted time-to-reach (S14), and outputs the forecasted result including the forecasted time-period-to-reach to at least one of the display 15 and the speaker 16 (S10). For example, the processor 11 outputs to the display 15 a signal for displaying an image based on the forecasted time-to-reach calculated in the process of S13. Alternatively, the processor 11 outputs to the display 15 a signal for displaying an image (for example, the image of icon 153 of FIG. 7) based on the forecasted time-period-to-reach calculated in the process of S14. Moreover, in the case where the audio notification using the speaker 16 is enabled by the icon 154, the processor 11 outputs, to the speaker 16, an audio signal based on the forecasted time-to-reach calculated in the process of S13 or an audio signal based on the forecasted time-period-to-reach calculated in the process of S14. After that, the processor 11 ends the present processes.

Note that in the case where the forecasted time-to-reach is calculated, at which the forecasted value of the time series data that are decreasing with the passage of time becomes less than the third threshold value, the CO2 concentration in the indoor space 20 is decreasing, and thus the processor 11 may send a notification of at least one of the forecasted time-to-reach and the forecasted time-period-to-reach until ventilation can be stopped using the display 15 and the speaker 16. On the other hand, in the case where the forecasted time-to-reach is calculated, at which the forecasted value of the time series data that are increasing with the passage of time exceeds the fourth threshold value, the CO2 concentration in the indoor space 20 is increasing, and thus the processor 11 may send a notification of at least one of the forecasted time-to-reach and the forecasted time-period-to-reach until the CO2 concentration reaches the value at which ventilation is necessary using the display 15 and the speaker 16.

As described above, the predictive sensor system 1 according to the embodiment 1 generates the forecasting model by joining the first model represented by the equation (1), which is suitable for forecasting in the first forecasting segment, and the second model represented by the equation (2), which is suitable for forecasting in the second forecasting segment that comes later than the first forecasting segment, using the hyperbolic function represented by the equation (3), and forecasts the change of time series data using the generated forecasting model.

Here, referring to the example of FIG. 3, in the case where the change of time series data is forecasted using only the first model, as represented by the graph G1, in the first forecasting segment in near future, the predictive sensor system 1 can forecast the values substantially similar to the actual measurement values. Whereas, in the second forecasting segment in distant future, it is difficult for the predictive sensor system 1 to forecast the values substantially similar to the actual measurement values. Further, in the case where the change of time series data is forecasted using only the second model, as represented by the graph G2, in the second forecasting segment in distant future, the predictive sensor system 1 can forecast the values substantially similar to the actual measurement values. whereas, in the first forecasting segment in near future, it is difficult for the predictive sensor system 1 to forecast the values substantially similar to the actual measurement values.

However, the predictive sensor system 1 according to the embodiment 1 enables to not only forecast the change of time series data in the first forecasting segment using the first model but also forecast the change of time series data in the second forecasting segment, which comes later than the first forecasting segment, using the second model. Accordingly, it becomes possible to accurately forecast the change of time series data over a long period of time.

Moreover, by using the forecasting model generated by joining the first model and the second model, the predictive sensor system 1 enables to obtain a forecasted result reflecting both the forecasted results of the first model and the second model in a forecasting segment in between the first forecasting segment and the second forecasting segment.

At the time of joining the first model and the second model, the predictive sensor system 1 weights the first model and the second model.

This enables the predictive sensor system 1 to adjust, using weighting, a period of time during which the forecasting is performed using the first model and a period of time during which the forecasting is performed using the second model. Moreover, in the forecasting segment in between the first forecasting segment and the second forecasting segment, the predictive sensor system 1 enables to obtain a forecasted result that reflects both the forecasted results of the first model and the second model with a ratio adjusted by weighting.

The predictive sensor system 1 adjusts the parameters of at least one of the first model and the second model based on the past time series data obtained by the sensor 14.

This enables the predictive sensor system 1 to perform highly accurate forecasting using the values of the parameters that fit to the change of time series data.

The predictive sensor system 1 causes the display 15 to display an image based on the forecasted result or causes the speaker 16 to output a sound based on the forecasted result.

This enables the predictive sensor system 1 to notify a user of information based on the forecasted result (for example, the remaining time period until ventilation of the indoor space 20 becomes necessary). Furthermore, because the predictive sensor system 1 enables to accurately forecast the change of time series data over a long period of time, it becomes possible to notify a user using the forecasted result not only for the first forecasting segment in near future but also for the second forecasting segment in distant future. Accordingly, for example, it becomes possible for a user to know the remaining time period until ventilation of the indoor space 20 becomes necessary with sufficient lead time.

### Embodiment 2

Referring to FIG. 10, a predictive sensor system 100 according to the embodiment 2 is described. FIG. 10 is a diagram illustrating a configuration of the predictive sensor system 100 according to the embodiment 2. In the following description, with regard to the predictive sensor system 100 according to the embodiment 2, only the parts different from the predictive sensor system 1 according to embodiment 1 are described.

As illustrated in FIG. 10, a forecasting system 1000 that forecasts the change of time series data includes the predictive sensor system 100 that has the function of serving as a server device, a monitoring device 200, and a notification device 300.

The predictive sensor system 100 includes a media reader device 13. The media reader device 13 receives a removable disk 5 that serves as a storage media, reads various programs and data stored in the removable disk 5, and outputs various programs and data stored in the storage device 12 to the removable disk 5.

For example, the media reader device 13 obtains the forecasting program 121 and the arithmetic data 122 stored in the removable disk 5 and outputs the obtained forecasting program 121 and the obtained arithmetic data 122 to the storage device 12. The storage device 12 stores the forecasting program 121 and the arithmetic data 122 that have been obtained from the media reader device 13. As described above, also in the predictive sensor system 100, without using the media reader device 13, various programs and data stored in advance in the storage device 12 may be used, or various programs and data downloaded from a network may be used.

The forecasting device 100 further includes a communication device 110. The communication device 110 is an example of the output device and is configured to transmit and receive data to and from each of the monitoring device 200 and the notification device 300 using communication via a network 500.

The monitoring device 200 includes a communication device 210, a first sensor 214A, and a second sensor 214B.

The communication device 210 is configured to transmit and receive data to and from the predictive sensor system 100 using communication via the network 500.

Each of the first sensor 214A and the second sensor 214B measures time series data that change as time passes, such as CO2 concentration, humidity, temperature, or the like. Note that the first sensor 214A may measure the same kind of time series data as the second sensor 214B or may measure a different kind of time series data than the second sensor 214B. Needless to say, the monitoring device 200 may measure time series data using only the first sensor 214A.

The notification device 300 includes a communication device 310, a display 315, and a speaker 316.

The communication device 310 is configured to transmit and receive data to and from each of the predictive sensor system 100 using communication via a network 500.

The display 315 displays various images such as an image based on the forecasted result of the change of time series data obtained from the predictive sensor system 100 and any other similar image.

The speaker 316 outputs various sounds such as a sound based on the forecasted result of the change of time series data obtained from the predictive sensor system 100 and any other similar sound.

In the forecasting system 1000 configured as described above, the monitoring device 200 transmits time series data obtained by each of the first sensor 214A and the second sensor 214B to the predictive sensor system 100 using the communication device 210. By executing the forecasting program 121, the predictive sensor system 100 forecasts the change of time series data based on the time series data obtained by the monitoring device 200 and transmits data based on the forecasted result to the notification device 300 using the communication device 110. Based on the forecasted result obtained from the predictive sensor system 100, the notification device 300 notifies a user of information based on the forecasted result using at least one of the display 315 and the speaker 316.

As described above, the predictive sensor system 100 according to the embodiment 2 forecasts the change of time series data based on the time series data obtained by the monitoring device 200 and outputs data based on the forecasted result to the notification device 300. This allows a user to obtain the forecasted result of the change of time series data while installing the predictive sensor system 100 at a location different from the sensor, the display, and the speaker. For example, the predictive sensor system 100 can be present in a form of cloud computing at a location different from the sensor, the display, and the speaker, which are installed in the indoor space 20.

Note that the forecasting system 1000 may include a plurality of monitoring devices 200, and the plurality of monitoring devices 200 may be connected to the predictive sensor system 100. Moreover, the forecasting system 1000 may include a device in which the monitoring device 200 and the notification device 300 are unified. Unifying the monitoring device 200 and the notification device 300 allows to share the communication device, and this enables to reduce the size and cut the cost of the forecasting system 1000.

The forecasting system 1000 may be configured using a closed network such as, for example, an intra-company LAN (Local Area Network), and this enables to structure an environment in which an environmental control in a company is performed only in the inside of the company.

### Embodiment 3

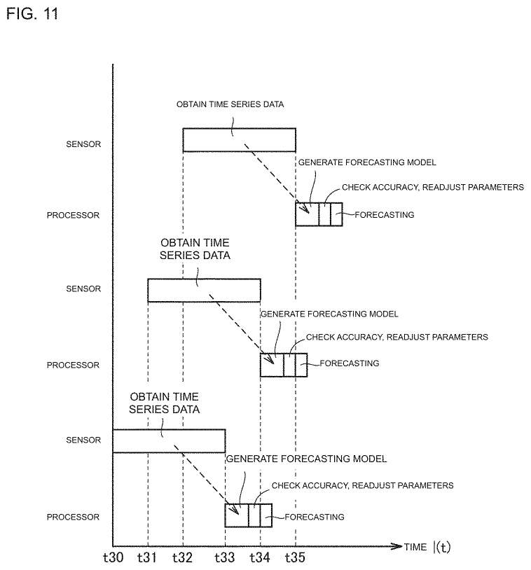

Referring to FIG. 11, a forecasting device according to the embodiment 3 is described. FIG. 11 is a diagram for illustrating timings of processes of the forecasting device according to the embodiment 3. In the following description, with regard to the forecasting device according to the embodiment 3, only the parts different from the predictive sensor system 1 according to embodiment 1 are described.

As illustrated in FIG. 11, in the forecasting device according to the embodiment 3, the processor 11 first forecasts the change of time series data based on time series data in one time segment and then forecasts the change of time series data based on time series data in the next time segment. At that time, the processor 11 may allow some data items to overlap between the time series data in one time segment and the time series data in the next time segment.

For example, when the sensor 14 obtains time series data over a time period from t30 to t33, the processor 11 generates the forecasting model after t33 based on the time series data (time series data from t30 to t33) and forecasts the change of time series data after t33 using the generated forecasting model.

When the sensor 14 obtains time series data over a time period from t31 to t34, the processor 11 generates the forecasting model after t34 based on the time series data (time series data from t31 to t34) and forecasts the change of time series data after t34 using the generated forecasting model.

When the sensor 14 obtains time series data over a time period from t32 to t35, the processor 11 generates the forecasting model after t35 based on the time series data (time series data from t32 to t35) and forecasts the change of time series data after t35 using the generated forecasting model.

In the example described above, some data items overlap between the time series data from t30 to t33 and the time series data from t31 to t34 and between the time series data from t30 to t33 and the time series data from t32 to t35.

As described above, at the time of forecasting the change of time series data, the forecasting device according to the embodiment 3 uses the time series data including data items that partially overlap with the time series data used in the previous forecasting, and this enables to forecast the change of time series data with a shorter cycle while retaining a sufficient amount of past time series data that are required to forecast the change of time series data.

Note that the sensor that obtains the time series data from t30 to t33, the sensor that obtains the time series data from t31 to t34, and the sensor that obtains the time series data from t32 to t35 may be mutually different sensors.

Moreover, as illustrated in FIG. 11, in the forecasting device according to the embodiment 3, the control device 11 may perform, after generating a forecasting model, a process for checking accuracy of the generated forecasting model. Further, the processor 11 may adjust the parameters (α, T₀) relating to the weighting.

Specifically, the processor 11 adjusts the parameters (α, T₀) relating to the weighting by checking the accuracy of the forecasting model using the function (distance function) represented by the following equation (7).

$$\begin{matrix}[ Math 7 ] & \\ F = {\sum }_{t} {( {C}_{R} ( t ) - {C}_{W} ( t ) )}^{2} & ( 7 )\end{matrix}$$

In the equation (7), C_{R}(t) is a function that indicates the actual measurement value at time t. Note that C_{R}(t) may alternatively be a function that indicates a corrected actual measurement value after performing the preprocessing. C_{W}(t) is a function of the forecasting model (for example, a hyperbolic function).

The processor 11 enables to optimize the forecasting model by calculating the parameters (α, T₀) that minimize the summation F of the function represented by the equation (7) and applying the calculated values to the function of the forecasting model.

As described above, the processor 11 enables to perform highly accurate forecasting in accordance with the change of time series data by adjusting the parameters (α, T₀) relating to the weighting at the time of joining the first model and the second model.

Note that when comparing the actual measurement values with the forecasted values obtained by the forecasting model using the distance function, the processor 11 may use a known distance function such as Euclidean distance, Manhattan distance, or the like. Moreover, the processor 11 may compare the actual measurement values with the forecasted values obtained by the forecasting model using a known Kalman filter.

### Embodiment 4

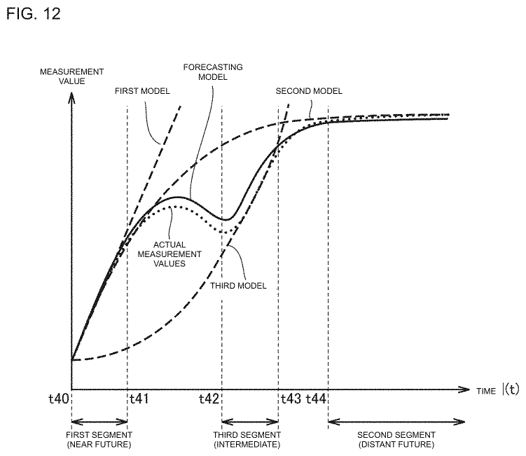

Referring to FIG. 12, a forecasting device according to the embodiment 4 is described. FIG. 12 is a diagram illustrating the change of time series data forecasted by the forecasting device according to the embodiment 4. In the following description, with regard to the forecasting device according to the embodiment 4, only the parts different from the predictive sensor system 1 according to embodiment 1 are described.

The forecasting device according to the embodiment 4 selects, in addition to the first model suitable for forecasting in the first forecasting segment in near future and the second model suitable for forecasting in the second forecasting segment in distant future, a third model suitable for forecasting in a third forecasting segment in between the first forecasting segment and the second forecasting segment. The third model is more suitable for forecasting the predicted data in the third forecasting segment compared with the first model and the second model. The forecasting device generates the forecasting model by joining the first model, the second model, and the third model.

As described above, the forecasting device according to the embodiment 4 generates the forecasting model by joining the first model suitable for forecasting in the first forecasting segment, the second model suitable for forecasting in the second forecasting segment, which comes later than the first forecasting segment, and the third model suitable for forecasting in the third forecasting segment in between the first forecasting segment and the second forecasting segment using the joining function, and forecasts the change of time series data using the generated forecasting model. Because of this, the forecasting device enables to not only forecast the change of time series data in the first forecasting segment using the first model but also forecast the change of time series data in a forecasting segment that comes later than the first forecasting segment using the second model or the third model. Accordingly, it becomes possible to accurately forecast the change of time series data over a long period of time.

Note that in the case where the forecasting model is generated using three or more models, the predictive sensor system 1 may be generated using the following equation.

For example, the predictive sensor system 1 joins the first model and the second model by using the following equation (8).

$$\begin{matrix}[ Math 8 ] & \\ {model}_{12} ( t ) = {model}_{1} ( t ) ( \frac{1 - tanh ( \frac{t - {T}_{12}}{{α}_{12}} )}{2} ) + {model}_{2} ( t ) ( \frac{1 + tanh ( \frac{t - {T}_{12}}{{α}_{12}} )}{2} ) & ( 8 )\end{matrix}$$

Moreover, by using the following equation (9), the predictive sensor system 1 joins the model generated by the equation (8) and the third model.

$$\begin{matrix}[ Math 9 ] & \\ {model}_{123} ( t ) = {model}_{12} ( t ) ( \frac{1 - tanh ( \frac{t - {T}_{123}}{{α}_{123}} )}{2} ) + {model}_{3} ( t ) ( \frac{1 + tanh ( \frac{t - {T}_{123}}{{α}_{123}} )}{2} ) & ( 9 )\end{matrix}$$

The predictive sensor system 1 repeats the calculations described by using the equation (8) and the equation (9) N times, which correspond to the number of the models, in accordance with the following equation (10).

$$\begin{matrix}[ Math 10 ] & \\ model \text{?} ( t ) = model \text{?} ( t ) ( \frac{1 - tanh ( \frac{t - T \text{?}}{\text{?}} )}{2} ) + {model}_{N} ( t ) ( \frac{1 + tanh ( \frac{t - T \text{?}}{\text{?}} )}{2} ) & ( 10 )\end{matrix}$$

$$\text{?} \text{indicates text missing or illegible when filed}$$

When the equations (10) are put together, the following equation (11) is obtained.

$$\begin{matrix}\{ Math 11 \} & \\ model \text{?} = \prod \text{?} ( \frac{1 - tanh ( \frac{t - T \text{?}}{\text{?}} )}{2} ) model \text{?} ( t ) + \sum \text{?} \prod \text{?} ( \frac{1 + \text{?} tanh ( \frac{t - T \text{?}}{\text{?}} )}{2} ) model \text{?} ( t ) & ( 11 )\end{matrix}$$

$$\text{?} \text{indicates text missing or illegible when filed}$$

Here, in the equation (11), the following equation (12) to equation (14) hold.

[Math 12]

(T₁ₖ,a₁ₖ)=[(T₁₂,a₁₂),(T₁₂₃,a₁₂₃), . . . ,(T_{12 . . . N},a_{12 . . . N})] (12)

$$\begin{matrix}[ Math 13 ] & \\ \text{?} & ( 13 )\end{matrix}$$

$$\text{?} \text{indicates text missing or illegible when filed}$$

[Math 14]

s_{12 . . . j}=1

s_{12 . . . j . . . l}=−1 l=j+1,j+2, . . . N (14)

Note that, of the functions included in the equation (11), the function represented by the following equation (15) is not limited to the first model that is suitable for forecasting in the first forecasting segment. In the case where a plurality of models are arranged in a time series manner, the model corresponding to the earliest forecasting segment may be applied to the function represented by the following equation (15).

$$\begin{matrix}[ Math 15 ] & \\ \prod \text{?} ( \frac{1 - tanh ( \text{?} )}{2} ) model \text{?} ( t ) & ( 15 )\end{matrix}$$

$$\text{?} \text{indicates text missing or illegible when filed}$$

Note that for each of a plurality of models to be used at the time of generating the forecasting model, such as the first model, the second model, and the third model, a known model capable of calculating an output (for example, CO2 concentration) for an input (for example, time) can be used. For example, each of the plurality of models may be at least one of a model that uses a neural network, a model that uses support vector regression, a model that uses logistic regression, a model that uses linear regression, a state space model, a model that uses Gaussian process regression, and a model that uses self-regression.

In general, the amount of calculation required for forecasting and the forecasting accuracy are in a trade-off relationship. That is to say, in the case where a forecasting model with a high degree of forecasting accuracy for a long period of time is to be generated, the amount of calculation tends to increase. On the other hand, as in the predictive sensor system 1 according to the embodiment, by respectively selecting a plurality of models, each of which only requires a small amount of calculation and has a short forecastable time period, for a plurality of forecasting segments and generating a single forecasting model by joining the plurality of models, it becomes possible to accurately forecast the change of time series data over a long period of time while limiting the increase in the amount of calculation.

Although a plurality of embodiments and modified examples are described, features of these plurality of embodiments and modified examples can be combined if appropriate unless such combination leads to contradiction.

It is to be understood that the embodiments described in the present disclosure are exemplary in all aspects and are not restrictive. It is intended that the scope of the present disclosure is determined by the claims, not by the description of the embodiments described above, and includes all variations which come within the meaning and range of equivalency of the claims.

FIG. 13 illustrates a block diagram of a more detailed diagram of electronic based components, including a programmable processor that serves to control processes of the various embodiments described herein. Moreover, the computer 805 includes more detailed features of the processor 11, of FIG. 2.

The control and sensor portions of the present disclosure may be embodied as a system, a method, and/or a computer program product. The computer program product may include a computer readable storage medium on which computer readable program instructions are recorded that may cause one or more processors to carry out aspects of the embodiment.

The computer readable storage medium may be a tangible device that can store instructions for use by an instruction execution device (processor). The computer readable storage medium may be, for example, but is not limited to, an electronic storage device, a magnetic storage device, an optical storage device, an electromagnetic storage device, a semiconductor storage device, or any appropriate combination of these devices. A non-exhaustive list of more specific examples of the computer readable storage medium includes each of the following (and appropriate combinations): flexible disk, hard disk, solid-state drive (SSD), random access memory (RAM), read-only memory (ROM), erasable programmable read-only memory (EPROM or Flash), static random access memory (SRAM), compact disc (CD or CD-ROM), digital versatile disk (DVD) and memory card or stick. A computer readable storage medium, as used in this disclosure, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described in this disclosure can be downloaded to an appropriate computing or processing device from a computer readable storage medium or to an external computer or external storage device via a global network (i.e., the Internet), a local area network, a wide area network and/or a wireless network. The network may include copper transmission wires, optical communication fibers, wireless transmission, routers, firewalls, switches, gateway computers and/or edge servers. A network adapter card or network interface in each computing or processing device may receive computer readable program instructions from the network and forward the computer readable program instructions for storage in a computer readable storage medium within the computing or processing device.

Computer readable program instructions for carrying out operations of the present disclosure may include machine language instructions and/or microcode, which may be compiled or interpreted from source code written in any combination of one or more programming languages, including assembly language, Basic, Fortran, Java, Python, R, C, C++, C# or similar programming languages. The computer readable program instructions may execute entirely on a user's personal computer, notebook computer, tablet, or smartphone, entirely on a remote computer or computer server, or any combination of these computing devices. The remote computer or computer server may be connected to the user's device or devices through a computer network, including a local area network or a wide area network, or a global network (i.e., the Internet). In some embodiments, electronic circuitry including, for example, programmable logic circuitry, field-programmable gate arrays (FPGA), or programmable logic arrays (PLA) may execute the computer readable program instructions by using information from the computer readable program instructions to configure or customize the electronic circuitry, in order to perform aspects of the present disclosure.

Aspects of the present disclosure are described herein with reference to flow diagrams and block diagrams of methods, apparatus (systems), and computer program products according to embodiments of the disclosure. It will be understood by those skilled in the art that each block of the flow diagrams and block diagrams, and combinations of blocks in the flow diagrams and block diagrams, can be implemented by computer readable program instructions.

The computer readable program instructions that may implement the systems and methods described in this disclosure may be provided to one or more processors (and/or one or more cores within a processor) of a general purpose computer, special purpose computer, or other programmable apparatus to produce a machine, such that the instructions, which execute via the processor of the computer or other programmable apparatus, create a system for implementing the functions specified in the flow diagrams and block diagrams in the present disclosure. These computer readable program instructions may also be stored in a computer readable storage medium that can direct a computer, a programmable apparatus, and/or other devices to function in a particular manner, such that the computer readable storage medium having stored instructions is an article of manufacture including instructions which implement aspects of the functions specified in the flow diagrams and block diagrams in the present disclosure.

The computer readable program instructions may also be loaded onto a computer, other programmable apparatus, or other device to cause a series of operational steps to be performed on the computer, other programmable apparatus or other device to produce a computer implemented process, such that the instructions which execute on the computer, other programmable apparatus, or other device implement the functions specified in the flow diagrams and block diagrams in the present disclosure.

FIG. 13 is a functional block diagram illustrating a networked system 800 of one or more networked computers and servers. In an embodiment, the hardware and software environment illustrated in FIG. 13 may provide an exemplary platform for implementation of the software and/or methods according to the present disclosure.

Referring to FIG. 13, a networked system 800 may include, but is not limited to, computer 805, network 810, remote computer 815, web server 820, cloud storage server 825 and computer server 830. In some embodiments, multiple instances of one or more of the functional blocks illustrated in FIG. 13 may be employed.

Additional detail of computer 805 is shown in FIG. 13. The functional blocks illustrated within computer 805 are provided only to establish exemplary functionality and are not intended to be exhaustive. And while details are not provided for remote computer 815, web server 820, cloud storage server 825 and computer server 830, these other computers and devices may include similar functionality to that shown for computer 805.

Computer 805 may be a personal computer (PC), a desktop computer, laptop computer, tablet computer, netbook computer, a personal digital assistant (PDA), a smart phone, or any other programmable electronic device capable of communicating with other devices on network 810.

Computer 805 may include processor 835, bus 837, memory 840, non-volatile storage 845, network interface 850, peripheral interface 855 and display interface 865. Each of these functions may be implemented, in some embodiments, as individual electronic subsystems (integrated circuit chip or combination of chips and associated devices), or, in other embodiments, some combination of functions may be implemented on a single chip (sometimes called a system on chip or SoC).

Processor 835 may be one or more single or multi-chip microprocessors, such as those designed and/or manufactured by Intel Corporation, Advanced Micro Devices, Inc. (AMD), Arm Holdings (Arm), Apple Computer, etc. Examples of microprocessors include Celeron, Pentium, Core i3, Core i5 and Core i7 from Intel Corporation; Opteron, Phenom, Athlon, Turion and Ryzen from AMD; and Cortex-A, Cortex-R and Cortex-M from Arm.

Bus 837 may be a proprietary or industry standard high-speed parallel or serial peripheral interconnect bus, such as ISA, PCI, PCI Express (PCI-e), AGP, and the like.

Memory 840 and non-volatile storage 845 may be computer-readable storage media. Memory 840 may include any suitable volatile storage devices such as Dynamic Random Access Memory (DRAM) and Static Random Access Memory (SRAM). Non-volatile storage 845 may include one or more of the following: flexible disk, hard disk, solid-state drive (SSD), read-only memory (ROM), erasable programmable read-only memory (EPROM or Flash), compact disc (CD or CD-ROM), digital versatile disk (DVD) and memory card or stick.

Program 848 may be a collection of machine readable instructions and/or data that is stored in non-volatile storage 845 and is used to create, manage and control certain software functions that are discussed in detail elsewhere in the present disclosure and illustrated in the drawings. In some embodiments, memory 840 may be considerably faster than non-volatile storage 845. In such embodiments, program 848 may be transferred from non-volatile storage 845 to memory 840 prior to execution by processor 835.

Computer 805 may be capable of communicating and interacting with other computers via network 810 through network interface 850. Network 810 may be, for example, a local area network (LAN), a wide area network (WAN) such as the Internet, or a combination of the two, and may include wired, wireless, or fiber optic connections. In general, network 810 can be any combination of connections and protocols that support communications between two or more computers and related devices.

Peripheral interface 855 may allow for input and output of data with other devices that may be connected locally with computer 805. For example, peripheral interface 855 may provide a connection to external devices 860. External devices 860 may include, for example, (1) user interface devices such as a keyboard, a mouse, a keypad, a touch screen, or input devices, (2) portable computer-readable storage media such as, for example, thumb drives, portable optical or magnetic disks, and memory cards, and (3) sensors, including temperature, pressure, fluid PH, and light sensors. Software and data used to practice embodiments of the present disclosure, for example, program 848, may be stored on such portable computer-readable storage media. In such embodiments, software may be loaded onto non-volatile storage 845 or, alternatively, directly into memory 840 via peripheral interface 855. Peripheral interface 855 may use an industry standard connection, such as RS-232 or Universal Serial Bus (USB), to connect with external devices 860.

Display interface 865 may connect computer 805 to display 870. Display 870 may be used, in some embodiments, to present a command line or graphical user interface to a user of computer 805. Display interface 865 may connect to display 870 using one or more proprietary or industry standard connections, such as VGA, DVI, DisplayPort and HDMI.

As described above, network interface 850, provides for communications with other computing and storage systems or devices external to computer 805. Software programs and data discussed herein may be downloaded from, for example, remote computer 815, web server 820, cloud storage server 825 and computer server 830 to non-volatile storage 845 through network interface 850 and network 810. Furthermore, the systems and methods described in this disclosure may be executed by one or more computers connected to computer 805 through network interface 850 and network 810. For example, in some embodiments the systems and methods described in this disclosure may be executed by remote computer 815, computer server 830, or a combination of the interconnected computers on network 810.

Power Supply 930 not only provides power for computer 805 but also optionally provides a power source for external devices 860. The power supply 930 may be implemented to draw power from an AC mains when the device is physically connected to an outlet. In this case, the power supply 930 may include an AC/DC converter that provides DC output for external devices, as well as to store charge in a rechargeable battery and/or capacitor. Alternatively, or additionally, the power supply 930 may receive a DC input, such as via a USB port. In this configuration the power supply 930 may provide DC power of various output voltages to external devices as well as AC power provided via a pulse code modulation-based DC to AC inverter.

Wireless transceiver 950 includes a radio frequency (RF) receiver and a RF transmitter that cooperate, under control of the processor 935 to exchange wireless communication signals with external devices. Non-limiting examples of the wireless communications may be local RF communications, such as by BLUETOOTH or Wi-Fi, or cellular transmission such as via 5G.

Data, datasets and/or databases employed in embodiments of the systems and methods described in this disclosure may be stored and or downloaded from remote computer 815, web server 820, cloud storage server 825 and computer server 830.

### REFERENCE SIGNS LIST

1, 100 Predictive Sensor System (Forecasting device)
5 Removable disk
11, 835 Processor
11A Generating part
11B Forecasting part
12 Storage device
13 Media reader device
14 Sensor
15, 315 Display
16, 316 Speaker
17 Output device
18, 840 Memory
20 Indoor space
21 Window
22 Door
110, 210, 310 Communication device
121 Forecasting program
122 Arithmetic data
123 Time series data
151, 153, 154 Icon
152 Graph
200 Monitoring device
214A First sensor
214B Second sensor
300 Notification device
500, 810 Network
800 Networked system
805 Computer
815 Remote Computer
820 Web Server
825 Cloud Storage Server
830 Computer Server
837 Bus
845 Non-volatile Storage
848 Program
850 Network Interface
855 Peripheral Interface
860 External Devices
865 Display Interface
870 Display
930 Power Supply
950 Wireless Transceiver
1000 Forecasting system

1. A predictive sensor system comprising:
a memory that has stored therein
time series data that is collected from a sensor that detects a parameter of an atmosphere in an inhabitable space, and
computer readable instructions; and
circuitry, which upon execution of the computer readable instructions, is configured to
apply the time series data to a trained predictive sensor model,
run the trained predictive sensor model on the time series data to generate predicted sensor measurement data for a future segment of time, and
generate a signal to cause an electronic device to generate a sensory output to alert an occupant in the inhabitable space of a level of the parameter of the atmosphere that corresponds with the predicted sensor measurement data, wherein
the trained predictive sensor model includes a first trained model and a second trained model,
the first trained model is trained to more closely match a portion of the predicted data in a first forecasting segment of time than the second trained model,
the second trained model is trained to more closely match another portion of the predicted sensor measurement data in a second forecasting segment of time than the first trained model, the second forecasting segment of time occurring later in time than the first forecasting segment of time, and
forecast an additional portion of the predicted data in another segment of time that occurs in between the first forecasting segment of time and the second forecasting segment of time based on the time series data and the trained predictive sensor model.

2. The predictive sensor system according to claim 1, wherein
the predictive sensor model is a weighted aggregation of the first model and the second model.

3. The predictive sensor system according to claim 2, wherein
the predictive sensor model includes an adjusted weighting parameter that relates to the time series data.

4. The predictive sensor system according to claim 1, wherein:
the memory has stored therein the trained predictive sensor model in addition to the time series data and the computer readable instructions that are executed by the circuitry to implement the trained predictive sensor model.

5. The predictive sensor system according to claim 1, further comprising:
the sensor that detects the parameter of the atmosphere in the inhabitable space.

6. The predictive sensor system according to claim 5, wherein
the sensor is a carbon dioxide (CO2) sensor and the parameter is CO2 concentration of the atmosphere in the inhabitable space.

7. The predictive sensor system according to claim 1, wherein
the circuitry is configured to output a forecasted time-to-reach at which an item of the predicted sensor measurement data is forecasted to reach a threshold value.

8. The predictive sensor system according to claim 1, wherein
the circuitry is configured to output a forecasted time-period-to-reach, which is a period of time that extends from a forecasting time point at which the predicted sensor measurement data are begun to be forecasted until a forecasted time-to-reach at which an item of the predicted sensor measurement data is forecasted to reach a threshold value.

9. The predictive sensor system according to claim 8, wherein
the circuitry is further configured to
calculate a first forecasted time-to-reach at a first forecasting time point, the first forecasted time-to-reach being a time at which an item of the predicted sensor measurement data is forecasted to reach the threshold value,
calculate a second forecasted time-to-reach at a second forecasting time point, the second forecasted time-to-reach being a later time at which another item of the predicted sensor measurement data is forecasted to reach the threshold value, the second forecasting time point being later in time than the first forecasting time point, and
under a condition a time period between the first forecasted time-to-reach and the second forecasted time-to-reach is greater than or equal to a predetermined time period, outputs a signal that indicates the forecasted time-to-reach has changed.

10. The predictive sensor system according to claim 1, further comprising:
the sensor, the being at least one of a CO2 sensor, a humidity sensor, a light level sensor, or a thermometer.

11. The predictive sensor system according to claim 10, further comprising:
the electronic device that is at least one of
a motor that is controllably actuated by the signal to control the motor to open at least one of a window, a door, or a vent, or
a switch that controllably operates a light, or a fan.

12. A predictive sensing method comprising:
storing, in a memory, time series data that is collected from a sensor that detects a parameter of an atmosphere in an inhabitable space; and
applying the time series data to a trained predictive sensor model implemented in circuitry that is configured by execution of computer readable instructions;
running the trained predictive sensor model on the time series data to generate predicted sensor measurement data for a future segment of time;
generating a signal to cause an electronic device to generate a sensory output to alert an occupant in the inhabitable space of a level of the parameter of the atmosphere that corresponds with the predicted sensor measurement data, wherein
the trained predictive sensor model includes a first trained model and a second trained model,
the first trained model is trained to more closely match a portion of the predicted data in a first forecasting segment of time than the second trained model,
the second trained model is trained to more closely match another portion of the predicted data in a second forecasting segment of time than the first trained model, the second forecasting segment of time being later in time than the first forecasting segment of time, and
the running includes forecasting an additional portion of the predicted sensor measurement data with the trained predictive sensor model in another segment of time in between the first forecasting segment of time and the second forecasting segment of time based on the time series data and the trained predictive sensor model.

13. The predictive sensing method according to claim 12, further comprising:
generating the predictive sensor model by weighting and aggregating the first trained model and the second trained model.

14. The predictive sensing method according to claim 13, wherein
the generating includes adjusting a weighting parameter that relates to the time series data.

15. The predictive sensing method according to claim 14, wherein
the running includes
adjusting a first parameter related to the weighting parameter, the first parameter is included in the first trained model and is based on the time series data, and
adjusting a second parameter related to the weighting parameter, the second parameter is included in the second trained model and is based on the time series data.

16. The predictive sensing method according to claim 13, wherein
the running includes adding the first trained model and the second trained model using a hyperbolic function.

17. The predictive sensing method according to claim 16, wherein
the hyperbolic function is represented by
$${C}_{W} ( t ) = {C}_{L} ( t ) * ( \frac{1 - tanh ( \frac{t - {T}_{0}}{α} )}{2} ) + {C}_{NL} ( t ) * ( \frac{1 + tanh ( \frac{t - {T}_{0}}{α} )}{2} )$$
wherein, C_{W} (t) is the hyperbolic function, C_{L}(t) is a characteristic function of the first trained model, C_{NL}(t) is a characteristic function of the second model, and α and T₀ are weighting parameters included during the aggregating.

18. The predictive sensing method according to claim 12, wherein
the trained predictive sensor model further includes a third trained model, and
the third trained model is trained to more closely match an addition portion of the predicted data in a third forecasting segment of time than either the first trained model or the second trained module in a third forecasting segment of time that occurs in between the first forecasting segment of time and the second forecasting segment of time.

19. The predictive sensing method according to claim 12, wherein
the first trained model characterizes a change in a first portion of the time series data over time in a linear form, and
the second trained model characterizes another change in a second portion of the time series data over time in a nonlinear form.

20. A non-transitory computer program product that has computer readable instructions stored therein that when executed by a processing circuitry causes the processing circuitry to implement a method, the method comprising:
storing, in a memory, time series data that is collected from a sensor that detects a parameter of an atmosphere in an inhabitable space; and
applying the time series data to a trained predictive sensor model implemented in circuitry that is configured by execution of computer readable instructions;
running the trained predictive sensor model on the time series data to generate predicted sensor measurement data for a future segment of time;
generating a signal to cause an electronic device to generate a sensory output to alert an occupant in the inhabitable space of a level of the parameter of the atmosphere that corresponds with the predicted sensor measurement data, wherein
the trained predictive sensor model includes a first trained model and a second trained model,
the first trained model is trained to more closely match a portion of the predicted data in a first forecasting segment of time than the second trained model,
the second trained model is trained to more closely match another portion of the predicted data in a second forecasting segment of time than the first trained model, the second forecasting segment of time being later in time than the first forecasting segment of time, and
the running includes forecasting an additional portion of the predicted sensor measurement data with the trained predictive sensor model in another segment of time in between the first forecasting segment of time and the second forecasting segment of time based on the time series data and the trained predictive sensor model.
